(19) United States
(12) Patent Application Publication
(10) Pub. No.: US 2018/0116582 A1
(43) Pub. Date: May 3, 2018

(54) ELASTICITY EVALUATION APPARATUS, ELASTICITY EVALUATION METHOD, AND ELASTICITY EVALUATION PROGRAM

(71) Applicant: FUJIFILM Corporation, Tokyo (JP)

(72) Inventor: Naoko YOSHIDA, Ashigarakami-gun (JP)

(73) Assignee: FUJIFILM Corporation, Tokyo (JP)

(21) Appl. No.: 15/856,929

(22) Filed: Dec. 28, 2017

Related U.S. Application Data
(63) Continuation of application No. PCT/JP2016/065316, filed on May 24, 2016.

(30) Foreign Application Priority Data
Jun. 30, 2015 (JP) 2015-130608

(51) Int. Cl. A61B 5/00

(52) U.S. Cl. CPC A61B 5/442; A61B 5/0077; A61B 5/7278; A61B 5/7264

(57) ABSTRACT

Provided are an elasticity evaluation apparatus, an elasticity evaluation method, and a non-transitory computer readable recording medium storing an elasticity evaluation program capable of easily evaluating elasticity of the skin with high accuracy. A skin index calculation part calculates existing amount of gloss portions of a skin in a captured image and existing amount of wrinkle portions of the skin in the captured image as skin evaluation indexes, and an elasticity evaluation part evaluates elasticity of a facial skin of a test subject on the basis of the skin evaluation indexes calculated by the skin index calculation part.

### CROSS-REFERENCE TO RELATED APPLICATIONS

This application is a Continuation of PCT International Application No. PCT/JP2016/065316 filed on May 24, 2016, which claims priority under 35 U.S.C § 119(a) to Japanese Patent Application No. 2015-130608 filed on Jun. 30, 2015. Each of the above application(s) is hereby expressly incorporated by reference, in its entirety, into the present application.

### BACKGROUND OF THE INVENTION

### 1. Field of the Invention

The present invention relates to an elasticity evaluation apparatus, an elasticity evaluation method, and an elasticity evaluation program, and particularly, to an elasticity evaluation apparatus, an elasticity evaluation method, and an elasticity evaluation program that evaluates elasticity of the skin on the basis of a captured image obtained by imaging the face of a test subject.

### 2. Description of the Related Art

In recent years, in the beauty field, as interest in elasticity of the skin that is one of the important conditions of a youthful skin increases, various methods for evaluating the elasticity of the skin have been studied. As one example of the methods, there is a method for evaluating visual elasticity when viewing the skin.

For example, JP2009-297064A discloses a method for evaluating visual elasticity using a sensual evaluation value obtained by evaluating a plurality of predetermined evaluation items, for example, such as moistness, gloss, elasticity, skin color, smoothness, color unevenness, brightness, using a plurality of panelists with respect to the face of a test subject.

JP2009-089999A discloses a method for binarizing an image obtained by imaging the face of a test subject from a front side using a camera in which a polarization filter is provided, extracting a white color reflecting region of a cheek portion, and performing particle diffraction, to thereby evaluate visual elasticity.

JP2008-093048A discloses a method for acquiring a brightness gradation image in a cheek region of a facial image of a test subject, and evaluating the visual elasticity on the basis of an area ratio in a predetermined gradation region of the image or a perimeter thereof.

JP2003-024306A discloses a method to evaluate elasticity of the skin by empirically extracting a predetermined image feature value based on Fourier transform, Wavelet transform, or the like from facial image data of a test subject on the basis of a known knowledge relating to a skin condition, and adding the result to a neural network that includes an evaluation value of elasticity of the skin acquired in advance by sensual evaluation of a plurality of panelists.

### SUMMARY OF THE INVENTION

However, in the evaluation method disclosed in JP2009-297064A, in order to enhance accuracy of visual elasticity obtained by sensual evaluation, data relating to tactile elasticity felt by touching a facial skin of a test subject, specifically, the amount of moisture of the facial skin of the test subject is also necessary. In order to obtain the data relating to the tactile elasticity, a contact-type elastometer is necessary.

Further, in the evaluation method disclosed in JP2009-089999A, it is necessary to provide a mechanical part such as a polarizing filter in an optical part or a lens part, and thus, it is not possible to easily evaluate visual elasticity of the skin.

Furthermore, in the evaluation method using only a physical feature, proposed in JP2008-093048A, it is not possible to obtain a sufficient correlation with a sense of elasticity of the skin felt when actually viewing the skin.

In the evaluation method disclosed in JP2003-024306A, it is necessary to prepare a lot of work or data in advance, such as an operation for constructing a neural network, and thus, it is not possible to easily evaluate visual elasticity of the skin.

The invention has been made in consideration of the problems in the related art, and an object of the invention is to provide an elasticity evaluation apparatus, an elasticity evaluation method, and a non-transitory computer readable recording medium storing an elasticity evaluation program capable of easily evaluating visual elasticity with high accuracy.

According to an aspect of the invention, there is provided an elasticity evaluation apparatus comprising: an image input part through which a captured image obtained by imaging a face of a test subject is input; a skin index calculation part that calculates existing amount of gloss portions of a skin in the captured image and existing amount of wrinkle portions of the skin in the captured image as skin evaluation indexes; and an elasticity evaluation part that evaluates visual elasticity of the face of the test subject on the basis of the skin evaluation indexes. Here, the skin index calculation part includes a gloss calculation part that calculates the existing amount of the gloss portions of the skin in the captured image, and a wrinkle calculation part that calculates the existing amount of the wrinkle portions of the skin in the captured image.

Here, the gloss calculation part may set a first evaluation region in the captured image, may detect the gloss portions in the first evaluation region, and may calculate a total area or an area ratio of the gloss portions as the existing amount of the gloss portions.

Further, the wrinkle calculation part may set second evaluation regions that extend from the nostrils to the mouth corners in the captured image, may detect wrinkle portions where the brightness in the second evaluation regions is lower than that in other regions, and may calculate a total area of the wrinkle portions, an area ratio of the wrinkle portions to the second evaluation regions, the shade of the wrinkle portions, or the length of the wrinkle portions in the second evaluation regions, as the existing amount of the wrinkle portions.

In addition, the gloss calculation part may set the first evaluation region in a skeletal portion of the face of the test subject, and may calculate the existing amount of the gloss portions in the first evaluation region on the basis of a brightness difference (ΔL*) and a saturation difference (ΔC*) between the first evaluation region and a portion where gloss is not easily generated in the face of the test subject.

The elasticity evaluation part may evaluate the visual elasticity on the basis of a linear sum of the existing amount of the gloss portions of the skin and the existing amount of the wrinkle portions of the skin with respect to a standard value of the visual elasticity that is calculated in advance by visually evaluating the faces of test subjects having different existing amounts of gloss portions of the skin and different existing amounts of wrinkle portions of the skin. The standard value of the visual elasticity is a value indicating the degree of visual elasticity.

The elasticity evaluation apparatus may further comprise a database that stores the linear sum of the existing amount of the gloss portions of the skin and the existing amount of the wrinkle portions of the skin with respect to the standard value, and the elasticity evaluation part may evaluate the visual elasticity with reference to the database on the basis of the existing amount of the gloss portions of the skin calculated by the gloss calculation part and the existing amount of the wrinkle portions of the skin calculated by the wrinkle calculation part.

According to another aspect of the invention, there is provided an elasticity evaluation method comprising: inputting a captured image obtained by imaging a face of a test subject; calculating existing amount of gloss portions of a skin in the captured image and existing amount of wrinkle portions of the skin in the captured image as skin evaluation indexes; and evaluating visual elasticity of the face of the test subject on the basis of the skin evaluation indexes.

According to still another aspect of the invention, there is provided a non-transitory computer readable recording medium storing an elasticity evaluation program for causing a computer to execute: a step of inputting a captured image obtained by imaging a face of a test subject; a step of calculating existing amount of gloss portions of a skin in the captured image and existing amount of wrinkle portions of the skin in the captured image as skin evaluation indexes; and a step of evaluating visual elasticity of the face of the test subject on the basis of the skin evaluation indexes.

According to the invention, it is possible to easily evaluate visual elasticity of a facial skin of a test subject with high accuracy.

### BRIEF DESCRIPTION OF THE DRAWINGS

FIG. 1 is a block diagram showing a configuration of an elasticity evaluation apparatus according to Embodiment 1 of the invention.

FIG. 2 is a block diagram showing a configuration of a skin index calculation part.

FIG. 3 is a diagram showing an evaluation region R1 and a standard region R1a set in the face of a test subject.

FIG. 4 is a diagram showing an evaluation region R2 set in the face of a test subject.

FIG. 5 is a diagram showing a correlation between a skin evaluation index and a sensual evaluation value with respect to skin elasticity, in which test results are plotted in an overlapping manner.

FIGS. 6A and 6B show a correlation relationship between a skin evaluation index and a sensual evaluation value with respect to skin elasticity, respectively, in which FIG. 6A shows a correlation between the existing amount of gloss and a sensual evaluation value, and FIG. 6B shows a correlation between the existing amount of wrinkles and a sensual evaluation value, respectively.

### DESCRIPTION OF THE PREFERRED EMBODIMENTS

Hereinafter, embodiments of the invention will be described with reference to the accompanying drawings.

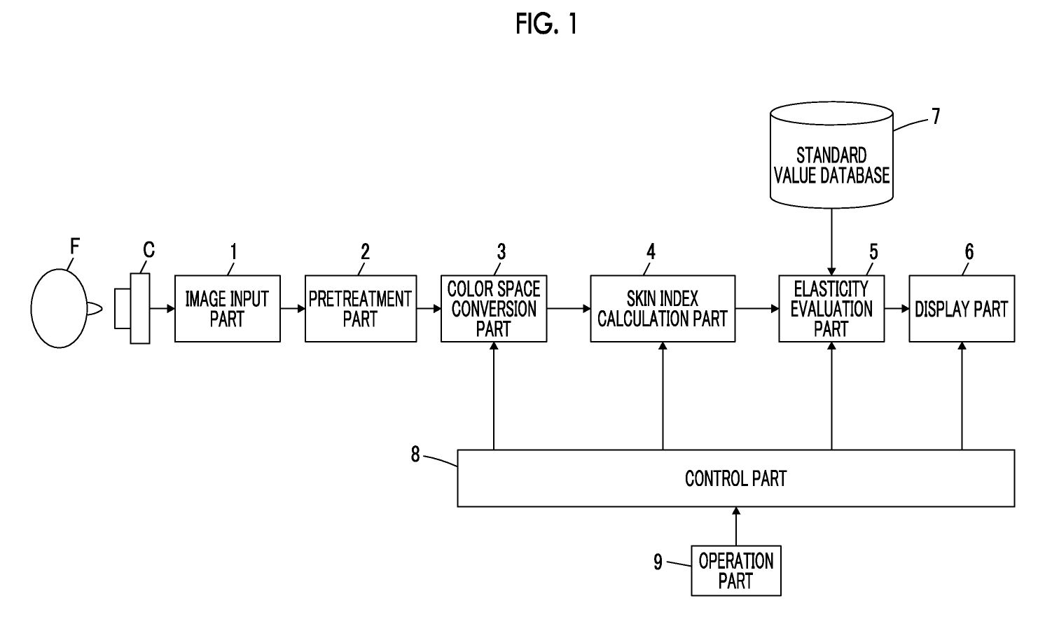

FIG. 1 shows a configuration of an elasticity evaluation apparatus according to Embodiment 1 of the invention. The elasticity evaluation apparatus is an apparatus that evaluates visual elasticity of the face F of a test subject using a captured image obtained by imaging the face F of the test subject using a camera C. The elastic evaluation apparatus includes an image input part 1 connected to the camera C, and a preprocessing part 2, a color space conversion part 3, a skin index calculation part 4, an elasticity evaluation part 5, and a display part 6 are sequentially connected to the image input part 1. Further, a standard value database 7 is connected to the elasticity evaluation part 5. In addition, a control part 8 is connected to the color space conversion part 3, the skin index calculation part 4, and the elasticity evaluation part 5. Further, an operation part 9 is connected to the control part 8.

Through the image input part 1, a captured image is input from the camera C that images the face F of the test subject. Here, it is assumed that the captured image input from the camera C has an RGB color space (color space of red, green, and blue). The camera C may be any type of camera capable of imaging the face F of the test subject, and for example, may be a digital camera that uses a charge-coupled device (CCD) or a complementary metal oxide semiconductor (CMOS), or a video camera. For example, an image captured using a mobile phone such as a smartphone may be used. In addition, the skin of the face F of the test subject may be a makeup skin, or may be a bare skin.

The preprocessing part 2 performs preprocessing such as light intensity correction, noise removal, and the like with respect to a captured image input from the image input part 1.

The color space conversion part 3 converts a color space of a captured image input from the preprocessing part 2 to generate a color space converted image. As the color space converted image, for example, an image obtained by converting the color space of the captured image into an L*a*b* color space, an LCH color space, an YCC color space, or the like may be used. In a case where the color space of the captured image is converted into the L*a*b* color space, a D65 light source may be used as a calculation light source. Further, the color space conversion part 3 divides the generated color space converted image into a brightness component and a color component to generate a brightness component image and a color component image, respectively. Specifically, in the case of the color space converted image having the L*a*b* color space, the brightness component represents an L* component, and the color component represents an a* component (complementary color component corresponding to red and green), a b* component (complementary component corresponding to yellow and blue), a C* component (saturation component), or the like.

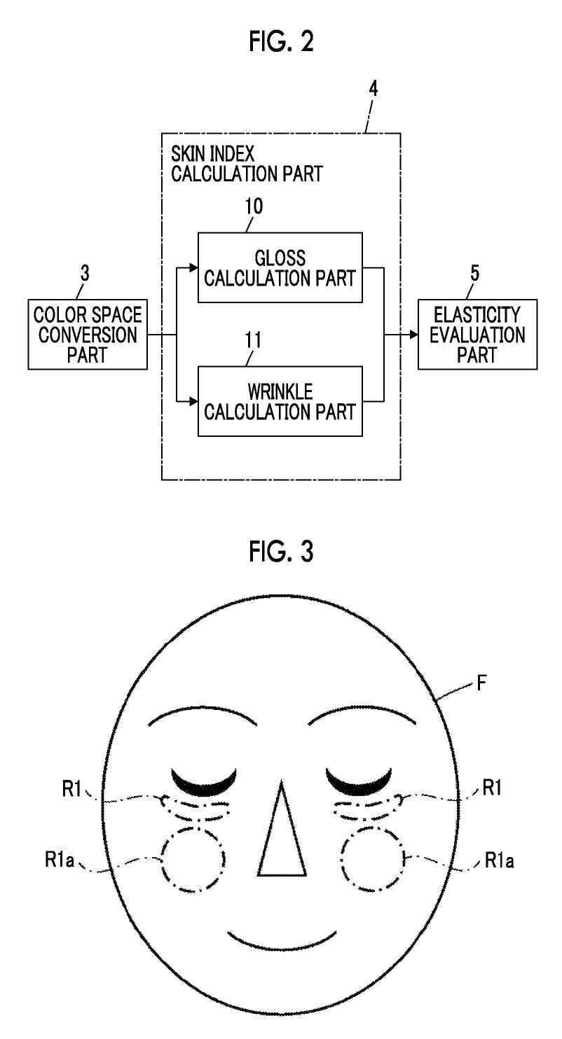

The skin index calculation part 4 includes a gloss calculation part 10 and a wrinkle calculation part 11, as shown in FIG. 2.

The skin index calculation part 4 is connected to the color space conversion part 3, receives an input of a color space converted image from the color space conversion part 3, and calculates a skin evaluation index for evaluating elasticity of the skin on the basis of the color space converted image. Here, the skin evaluation index refers to an evaluation index that is in common with an index for evaluating elasticity of the skin, which evaluates visual elasticity according to a sense when generally viewing the skin.

The skin index calculation part 4 outputs the existing amount of gloss portions of the skin calculated by the gloss calculation part 10, and the existing amount of wrinkle portions of the skin calculated by the wrinkle calculation part 11 to the elasticity evaluation part 5, as the skin evaluation indexes.

The standard value database 7 stores physical features due to visual elasticity, for example, the existing amounts of stains, wrinkles, pores and sags, and a relationship of the existing amount of gloss portions of the skin and the existing amount of wrinkle portions of the skin with respect to a standard value of visual elasticity obtained in advance by visually evaluating the faces of a plurality of test subjects having different brightnesses and tones of the skins. For example, a linear sum of the existing amount of gloss portions of the skin and the existing amount of wrinkle portions of the skin may be calculated, and a function indicating the standard value of the visual elasticity with respect to the linear sum may be stored.

The elasticity evaluation part 5 evaluates elasticity of the face F of the test subject on the basis of the skin evaluation index calculated by the skin index calculation part 4.

Specifically, the elasticity evaluation part 5 calculates an evaluation value of visual elasticity with reference to the standard value database 7 on the basis of the existing amount of gloss portions of the skin calculated by the gloss calculation part 10 and the existing amount of wrinkle portions of the skin calculated by the wrinkle calculation part 11. For example, the elasticity evaluation part 5 is able to calculate the evaluation value the visual elasticity on the basis of a function indicating the standard value of the visual elasticity with respect to a linear sum of the existing amount of gloss portions of the skin and the existing amount of wrinkle portions of the skin stored in the standard value database 7.

Here, the elasticity represents an elastic skin, and the elasticity evaluation part 5 evaluates visual elasticity indicating elasticity felt when generally viewing the face F of the test subject.

The display part 6 includes a display device such as a liquid crystal display (LCD), for example, and displays an evaluation result of the visual elasticity evaluated by the elasticity evaluation part 5.

The operation part 9 is a unit through which an operator performs an input operation of information, and may be formed by a keyboard, a mouse, a track board, a touch panel, or the like.

The control part 8 controls respective parts in the elasticity evaluation apparatus on the basis of various command signals input through the operation part 9 from an operator.

The color space conversion part 3, the skin index calculation part 4, the elasticity evaluation part 5 and the control part 8 are configured by a central processing unit (CPU) and an operation program for causing the CPU to execute various operations, but may be configured by a digital circuit. Further, a memory may be connected to the CPU through a signal line such as a bus, for example. A color space converted image generated by the color space conversion part 3, an image generated by the skin index calculation part 4, an evaluation result of elasticity calculated by the elasticity evaluation part 5, or the like may be stored in the memory, and the images and the evaluation result of the elasticity stored in the memory may be displayed on the display part 6 under the control of the control part 8.

Next, the skin index calculation part 4 will be described in detail.

As shown in FIG. 2, the skin index calculation part 4 includes the gloss calculation part 10 and the wrinkle calculation part 11 that are respectively connected to the color space conversion part 3 and the elasticity evaluation part 5.

The gloss calculation part 10 calculates the existing amount of gloss portions in the skin as a skin evaluation index. Here, the gloss portions are generated in skeletal portions of the face, specifically, cheek bones, nasal muscles, or the like, and when a portion having a high reflectivity of light is referred as a shine portion and a portion having a low reflectivity of light is referred to as a mat feeling portion in a facial skin, the gloss portions have a reflectivity of light between the bright portion and the mat feeling portion.

The gloss calculation part 10 sets evaluation regions R1 with respect to a brightness component image and a color component image generated by the color space conversion part 3. The evaluation regions R1 may be set with respect to outline portions of the face such as cheek bones or nasal muscles. Subsequently, the gloss calculation part 10 detects gloss portions from the evaluation regions R1 on the basis of a brightness value and a color component value and calculates, for example, the existing amount of gloss portions on the basis of a total area (the number of pixels) of the gloss portions in the evaluation regions R1 or an area ratio of the gloss portions to the evaluation regions R1.

The wrinkle calculation part 11 calculates the existing amount of wrinkle portions in the skin as a skin evaluation index. Here, the wrinkle portions have a brightness component value and a color component value that are locally changed and have an elongated shape that extends in a predetermined direction.

Specifically, the wrinkle calculation part 11 sets evaluation regions R2 that extend from the nostrils to mouth corners with respect to a brightness component image generated by the color space conversion part 3. Subsequently, the wrinkle calculation part 11 detects wrinkle portions where the brightness is low in the evaluation regions R2, and calculates, for example, a total area of the wrinkle portions in the evaluation regions R2, an area ratio of the wrinkle portions to the evaluation regions R2, the shade of the wrinkle portions based on the strength of the brightness, or the length of the wrinkle portions in the evaluation regions R2, as a skin evaluation index.

Next, an operation of Embodiment 1 will be described.

First, a captured image obtained by imaging the face F of the test subject using the camera C is input to the preprocessing part 2 through the image input part 1 of the elasticity evaluation apparatus from the camera C, as shown in FIG. 1. The captured image is subjected to preprocessing such as light source correction or noise removal, and then, is output to the color space conversion part 3 from the preprocessing part 2. Then, a color space of the captured image is converted into an L*a*b* color space, for example, by the color space conversion part 3, so that a color space converted image is generated. Further, the color space conversion part 3 extracts a brightness component and a color component from the color space converted image, to thereby generate a brightness component image and a color component image, respectively. For example, an L* component image may be generated as the brightness component image, and a C* component image, an a* component image, a b* component image, or the like may be generated as the color component image. In the L*a*b* color space, saturation C* is calculated according to the following expression.

C*=\sqrt{(a*²)+(b*²)}

The color space conversion part 3 outputs the generated L* component image, C* component image, a* component image, and b* component image to the gloss calculation part 10 and the wrinkle calculation part 11 of the skin index calculation part 4, respectively.

The gloss calculation part 10 sets the evaluation regions R1 in the face F of the test subject with respect to the L* component image and the C* component image input from the color space conversion part 3, and detects gloss portions from the set evaluation regions R1.

Specifically, the gloss calculation part 10 sets the evaluation regions R1 in regions where gloss is easily generated in the face F of the test subject, and sets standard regions R1a in regions where gloss is not easily generated. The evaluation regions R1 may be set in outline portions of the face such as cheek bones, as shown in FIG. 3, for example. Further, the standard regions R1a may be set in cheek central portions.

The gloss calculation part 10 calculates an average value of L* components and an average value of C* components with respect to the evaluation regions R1 and the standard regions R1a.

A person feels the presence or absence, and the strength of gloss in a human skin while viewing brilliance (brightness) reflected on the front surface of the skin. A person feels gloss more easily as a difference between brightness in a region where gloss is not easily generated and brightness in a region where gloss is easily generated becomes large, and recognizes brilliance reflected on the skin as more gloss, as tone of the brilliance becomes little. Thus, it may be considered that an L* component value and a C* component value in the evaluation regions R1 calculated by the gloss calculation part 10 form one index indicating a change of gloss.

Further, the gloss calculation part 10 creates an ΔL* component image obtained by subtracting the average value of the L* components in the standard regions R1a from the average value of the L* components in the evaluation regions R1, and creates an ΔC* component image obtained by subtracting the average value of the C* components in the standard regions R1a from the average value of the C* components in the evaluation regions R1.

Subsequently, the gloss calculation part 10 calculates the existing amount of gloss portions in the evaluation regions R1 on the basis of the ΔL* component image and the ΔC* component image as a skin evaluation index.

The gloss calculation part 10 detects gloss portions from the evaluation regions R1 of the ΔL* component image and the ΔC* component image on the basis of a predetermined threshold value that is set in advance. Specifically, the gloss calculation part 10 may detect portions in which the ΔL* component value is smaller than 3.0 and the ΔC* component value exceeds 2.0 as the gloss portions.

Further, for example, the gloss calculation part 10 calculates the skin evaluation index on the basis of a linear sum of the ΔL* component value and the ΔC* component value in the gloss portions detected in the evaluation regions R1. Specifically, the gloss index may be calculated using an expression “gloss index=α×ΔL* component value+β×ΔC* component value+γ”. Here, it is preferable that γ is a constant, a is −4.0 to −6.0, and β is 2.0 to 4.0, and it is more preferable that a is −5.4 and β is 2.4.

The gloss calculation part 10 outputs the calculated skin evaluation index to the elasticity evaluation part 5.

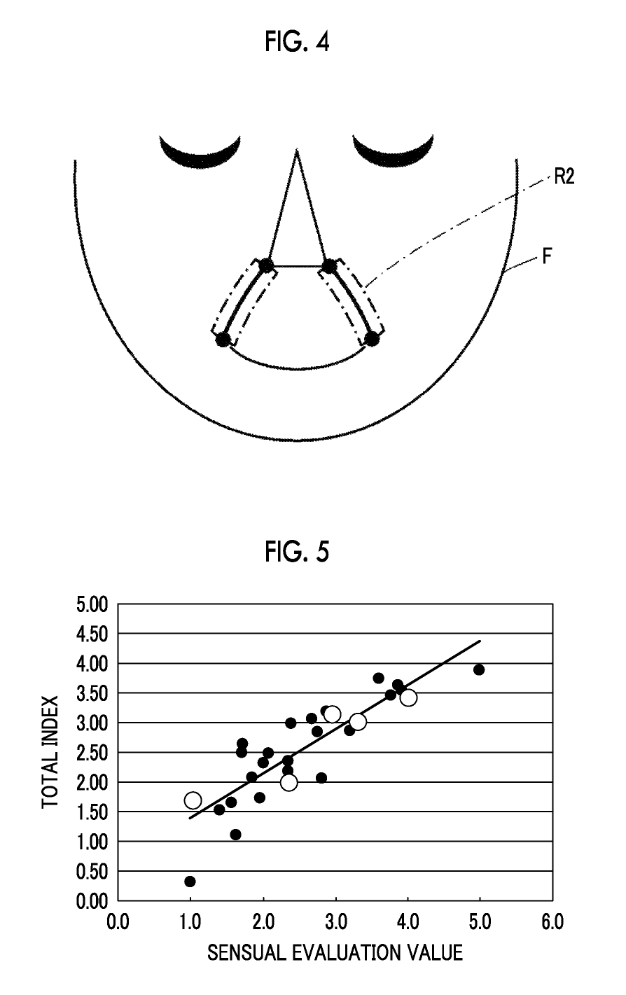

The wrinkle calculation unit 11 sets the evaluation regions R2 that extend from the nostrils to the mouth corners, as shown in FIG. 4, with respect to the brightness component image or the color component image input from the color space conversion part 3, and detects wrinkle portions from the evaluation regions R2. Further, the wrinkle calculation part 11 sets standard regions R2a in the vicinity of the evaluation region R2, and calculates an average value of L* components in the standard regions R2a.

Subsequently, the wrinkle calculation part 11 creates an ΔL* component image obtained by subtracting the average value of the L* components in the standard regions R2a from the L* component value in the evaluation regions R2, and detects wrinkle portions from the evaluation regions R2 of the ΔL* component image on the basis of a predetermined threshold value that is set in advance. For example, portions in which the ΔL* component value is smaller than 10 in the evaluation region R2 may be detected as the wrinkle portions.

Further, the wrinkle calculation part 11 calculates a value obtained by standardizing a total area of wrinkle portions detected in the evaluation regions R2 as a skin evaluation index, for example. Here, the wrinkle portions detected in the evaluation regions R2 are so-called smile lines. When generally viewing the face F of the test subject, the wrinkle portions give an impression as rough portions where dark shadows are generated. As the total area becomes smaller, visual elasticity of the face F of the test subject is felt more strongly.

The wrinkle calculation part 11 outputs the calculated skin evaluation index to the elasticity evaluation part 5.

In this way, two skin evaluation indexes that are respectively calculated by the gloss calculation part 10 and the wrinkle calculation part 11 are input to the elasticity evaluation part 5.

The standard value database 7 may calculate a linear sum of the existing amount of gloss portions and the existing amount of wrinkle portions, which are determined in advance by visually evaluating the faces of a plurality of test subjects having different existing amounts of gloss portions and existing amounts of wrinkle portions, and may store a function indicating a standard value of visual elasticity with respect to the linear sum.

Before calculating the linear sum, it is preferable to standardize the existing amount of gloss portions and the existing amount of wrinkle portions. Specifically, ΔL* component images and ΔC* component images of persons of various ages are acquired in advance, a maximum value of ΔL* components is regulated as an ΔL* component maximum value, and a maximum value of ΔC* components is regulated as an ΔC* component maximum value. Further, by dividing the ΔL* components of the images of the test subjects by the regulated ΔL* component maximum value, and by dividing the ΔC* components of the images of the test subjects by the ΔC* component maximum value, each standardization is performed. Similarly, total wrinkle areas of persons of various ages are individually acquired in advance, and the largest area among them is regulated as a maximum wrinkle amount. Further, by dividing the total area of the wrinkles of the images of the test subjects by the regulated maximum wrinkle amount, the standardization is performed.

The elasticity evaluation part 5 evaluates visual elasticity of the face of a test subject on the basis of a skin evaluation index obtained from the existing amount of gloss portions and the existing amount of wrinkle portions.

Specifically, the elasticity evaluation part 5 calculates an evaluation value of the visual elasticity with reference to the standard value database 7 on the basis of the existing amount of gloss portions calculated by the gloss calculation part 10 and the existing amount of wrinkle portions calculated by the wrinkle calculation part 11. For example, the elasticity evaluation part 5 is able to evaluate the visual elasticity on the basis of a function indicating a standard value of the visual elasticity with respect to a linear sum of the existing amount of gloss portions and the existing amount of wrinkle portions of the skin stored in the standard value database 7.

According to the above-described embodiment, since elasticity is objectively evaluated according to a sense when generally viewing the face F of the test subject using two indexes calculated by the skin index calculation part 4, it is possible to obtain an evaluation result having a high correlation with evaluation of elasticity through sensual evaluation.

The evaluation of skin elasticity as in the embodiment may be executed by an elasticity evaluation program to cause a computer that includes input means, a CPU, a memory, and the like to function. That is, the elasticity evaluation program causes the computer to function so that the image input part 1 acquires a captured image obtained by imaging the face of a test subject and the CPU executes the preprocessing part 2, the color space conversion part 3, the skin index calculation part 4, and the elasticity evaluation part 5 to evaluate elasticity with respect to the face of the test subject on the basis of the acquired captured image.

In the above-described embodiment, the skin index calculation part 4 calculates two skin evaluation indexes from the existing amount of gloss portions of the skin and the existing amount of wrinkle portions of the skin, but the invention is not limited thereto.

In the above-described embodiment, in a case where a correlation is calculated between two skin evaluation index and sensual evaluation values in the standard value database 7, facial images of a plurality of test subjects having different physical features due to visual elasticity, for example, different stains, wrinkles, pores and sags, or brightnesses and tones of the skins are used, but the invention is not limited thereto. A plurality of facial images of one test subject obtained by variously changing physical features thereof may be used.

### Example

In reality, an example in which elasticity of the face of a test subject is evaluated using an elasticity evaluation apparatus is shown.

In this example, 27 images subjected to a process of variously changing physical features of the skin due to visual elasticity with respect to a facial image of a certain test subject were prepared. The 27 images were divided into (A) 22 images (model creation data) used for creating a graph indicating a correlation between two skin evaluation indexes and sensual evaluation values in the elasticity evaluation apparatus, for example, a linear function, and (B) five images (test data) used for confirming reliability of the created graph. First, with respect to the 22 images grouped as the (A) model creation data, using the elasticity evaluation apparatus, evaluation of visual elasticity of the skin was performed, and also, sensual evaluation of visual elasticity was performed when 20 observers generally viewed the face F of the test subject.

In FIG. 5, total index values calculated by linearly summing two skin evaluation indexes obtained using the elasticity evaluation apparatus, that is, the existing amount of gloss portions and the existing amount of wrinkle portions are plotted as “black circles” with respect to sensual evaluation values. Specifically, total index=0.031×gloss existing amount−0.041×wrinkle portion existing amount+γ. Here, γ is a constant.

Further, here, the sensual evaluation value is an average value obtained by evaluating visual elasticity at five stages through sensual evaluation of 20 observers. As the value comes close to 1, it is evaluated that elasticity is not present, and as the value comes close to 5, it is evaluated that elasticity is present.

Then, with respect to five images selected as the test data (B), a total index value calculated by linearly summing two skin evaluation indexes, that is, the existing amount of gloss portions and the existing amount of wrinkle portions using the elasticity evaluation apparatus was calculated, and in the same way as the above-described method, the sensual evaluation values were calculated. The results are plotted as “white circles” in FIG. 5.

### Comparative Example

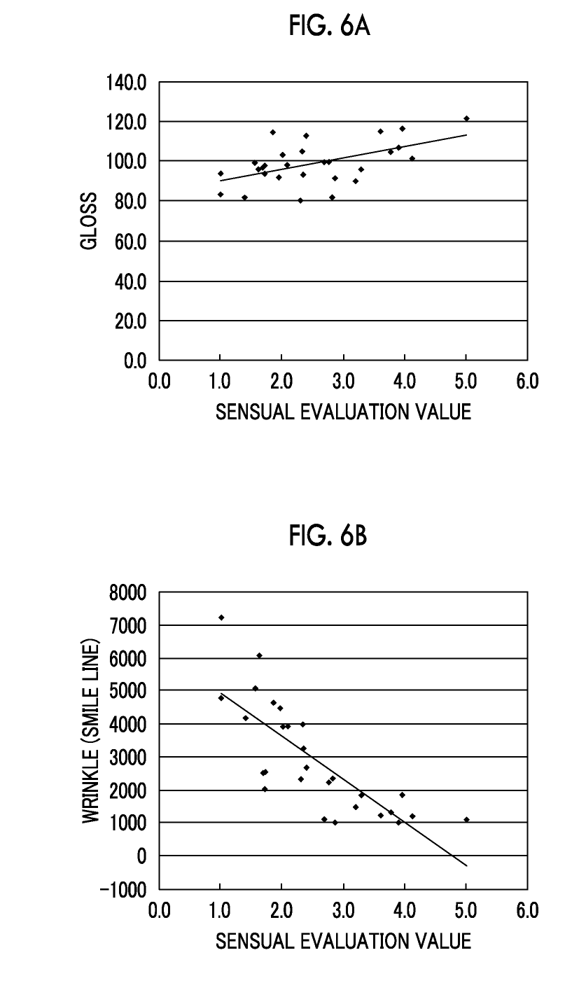

FIG. 6A is a diagram showing results obtained by calculating the existing amount of gloss portions using an elasticity evaluation apparatus with respect to a plurality of images obtained by variously changing physical features of the skin due to visual elasticity, performing sensual evaluation of elasticity, and plotting the existing amount of gloss portions with respect to a sensual evaluation value, similar to Example.

FIG. 6B is a diagram showing results obtained by calculating the existing amount of wrinkle portions (smile lines) using an elasticity evaluation apparatus with respect to a plurality of images obtained by variously changing physical features of the skin due to visual elasticity, performing sensual evaluation of elasticity, and plotting the existing amount of wrinkle portions with respect to a sensual evaluation value, similar to Example.

On the basis of FIG. 5, as a result of calculation of a correlation between a total index value and a sensual evaluation value, a correlation coefficient R² was 0.7297. Further, in the figure, “white circles” indicating test results for confirming reliability of a graph indicating the correlation between the created total index values and sensual evaluation values were plotted on the graph or in the vicinity of the graph. From this result, it can be understood that the reliability of the elasticity evaluation based on two skin evaluation indexes is high.

With respect to FIG. 6A, as a result of calculation of a correlation between a gloss evaluation index and a sensual evaluation value, a correlation coefficient R² was 0.2979. Further, with respect to FIG. 6B, as a result of calculation of a correlation between a wrinkle evaluation index and a sensual evaluation value, a correlation coefficient R² was 0.612.

From this result, it can be understood that when the elasticity of the skin is evaluated on the basis of only each skin evaluation index, it is not possible to accurately evaluate elasticity of the skin, and in order to appropriately evaluate elasticity of a facial skin, it is necessary to evaluate the elasticity of the skin by combination of two skin evaluation indexes, that is, the existing amount of gloss portions and the existing amount of wrinkle portions.

### EXPLANATION OF REFERENCES

1: image input part
2: preprocessing part
3: color space conversion part
4: skin index calculation part
5: elasticity evaluation part
6: display part
7: standard value database
8: control part
9: operation part
10: gloss calculation part
11: wrinkle calculation part
R1, R2: evaluation region
R1a: standard region
F: face
C: camera

What is claimed is:

1. An elasticity evaluation apparatus comprising:
an image input part through which a captured image obtained by imaging a face of a test subject is input;
a skin index calculation part that calculates existing amount of gloss portions of a skin in the captured image and existing amount of wrinkle portions of the skin in the captured image as skin evaluation indexes; and
an elasticity evaluation part that evaluates visual elasticity of the face of the test subject on the basis of the skin evaluation indexes,
wherein the skin index calculation part includes a gloss calculation part that calculates the existing amount of the gloss portions of the skin in the captured image, and a wrinkle calculation part that calculates the existing amount of the wrinkle portions of the skin in the captured image.

2. The elasticity evaluation apparatus according to claim 1,
wherein the gloss calculation part sets a first evaluation region in the captured image, detects the gloss portions in the first evaluation region, and calculates a total area or an area ratio of the gloss portions as the existing amount of the gloss portions.

3. The elasticity evaluation apparatus according to claim 1,
wherein the wrinkle calculation part sets second evaluation regions that extend from the nostrils to mouth corners in the captured image, detects wrinkle portions where the brightness in the second evaluation regions is lower than that in other regions, and calculates a total area of the wrinkle portions, an area ratio of the wrinkle portions to the second evaluation regions, the shade of the wrinkle portions, or the length of the wrinkle portions in the second evaluation regions, as the existing amount of the wrinkle portions.

4. The elasticity evaluation apparatus according to claim 2,
wherein the wrinkle calculation part sets second evaluation regions that extend from the nostrils to mouth corners in the captured image, detects wrinkle portions where the brightness in the second evaluation regions is lower than that in other regions, and calculates a total area of the wrinkle portions, an area ratio of the wrinkle portions to the second evaluation regions, the shade of the wrinkle portions, or the length of the wrinkle portions in the second evaluation regions, as the existing amount of the wrinkle portions.

5. The elasticity evaluation apparatus according to claim 1,
wherein the gloss calculation part sets the first evaluation region in a skeletal portion of the face of the test subject, and calculates the existing amount of the gloss portions in the first evaluation region on the basis of a brightness difference (ΔL*) and a saturation difference (ΔC*) between the first evaluation region and a portion where gloss is not easily generated in the face of the test subject.

6. The elasticity evaluation apparatus according to claim 2,
wherein the gloss calculation part sets the first evaluation region in a skeletal portion of the face of the test subject, and calculates the existing amount of the gloss portions in the first evaluation region on the basis of a brightness difference (ΔL*) and a saturation difference (ΔC*) between the first evaluation region and a portion where gloss is not easily generated in the face of the test subject.

7. The elasticity evaluation apparatus according to claim 1,
wherein the elasticity evaluation part evaluates the visual elasticity on the basis of a linear sum of the existing amount of the gloss portions of the skin and the existing amount of the wrinkle portions of the skin with respect to a standard value of the visual elasticity that is calculated in advance by visually evaluating the faces of test subjects having different existing amounts of gloss portions of the skin and different existing amounts of wrinkle portions of the skin.

8. The elasticity evaluation apparatus according to claim 2,
wherein the elasticity evaluation part evaluates the visual elasticity on the basis of a linear sum of the existing amount of the gloss portions of the skin and the existing amount of the wrinkle portions of the skin with respect to a standard value of the visual elasticity that is calculated in advance by visually evaluating the faces of test subjects having different existing amounts of gloss portions of the skin and different existing amounts of wrinkle portions of the skin.

9. The elasticity evaluation apparatus according to claim 3,
wherein the elasticity evaluation part evaluates the visual elasticity on the basis of a linear sum of the existing amount of the gloss portions of the skin and the existing amount of the wrinkle portions of the skin with respect to a standard value of the visual elasticity that is calculated in advance by visually evaluating the faces of test subjects having different existing amounts of gloss portions of the skin and different existing amounts of wrinkle portions of the skin.

10. The elasticity evaluation apparatus according to claim 4,
wherein the elasticity evaluation part evaluates the visual elasticity on the basis of a linear sum of the existing amount of the gloss portions of the skin and the existing amount of the wrinkle portions of the skin with respect to a standard value of the visual elasticity that is calculated in advance by visually evaluating the faces of test subjects having different existing amounts of gloss portions of the skin and different existing amounts of wrinkle portions of the skin.

11. The elasticity evaluation apparatus according to claim 7, further comprising:
a database that stores the linear sum of the existing amount of the gloss portions of the skin and the existing amount of the wrinkle portions of the skin with respect to the standard value,
wherein the elasticity evaluation part evaluates the visual elasticity with reference to the database on the basis of the existing amount of the gloss portions of the skin calculated by the gloss calculation part and the existing amount of the wrinkle portions of the skin calculated by the wrinkle calculation part.

12. The elasticity evaluation apparatus according to claim 8, further comprising:
a database that stores the linear sum of the existing amount of the gloss portions of the skin and the existing amount of the wrinkle portions of the skin with respect to the standard value,
wherein the elasticity evaluation part evaluates the visual elasticity with reference to the database on the basis of the existing amount of the gloss portions of the skin calculated by the gloss calculation part and the existing amount of the wrinkle portions of the skin calculated by the wrinkle calculation part.

13. The elasticity evaluation apparatus according to claim 9, further comprising:
a database that stores the linear sum of the existing amount of the gloss portions of the skin and the existing amount of the wrinkle portions of the skin with respect to the standard value,
wherein the elasticity evaluation part evaluates the visual elasticity with reference to the database on the basis of the existing amount of the gloss portions of the skin calculated by the gloss calculation part and the existing amount of the wrinkle portions of the skin calculated by the wrinkle calculation part.

14. The elasticity evaluation apparatus according to claim 10, further comprising:
a database that stores the linear sum of the existing amount of the gloss portions of the skin and the existing amount of the wrinkle portions of the skin with respect to the standard value,
wherein the elasticity evaluation part evaluates the visual elasticity with reference to the database on the basis of the existing amount of the gloss portions of the skin calculated by the gloss calculation part and the existing amount of the wrinkle portions of the skin calculated by the wrinkle calculation part.

15. An elasticity evaluation method comprising:
inputting a captured image obtained by imaging a face of a test subject;
calculating existing amount of gloss portions of a skin in the captured image and existing amount of wrinkle portions of the skin in the captured image as skin evaluation indexes; and
evaluating visual elasticity of the face of the test subject on the basis of the skin evaluation indexes.

16. A non-transitory computer readable recording medium storing an elasticity evaluation program for causing a computer to execute:
a step of inputting a captured image obtained by imaging a face of a test subject;
a step of calculating existing amount of gloss portions of a skin in the captured image and existing amount of wrinkle portions of the skin in the captured image as skin evaluation indexes; and
a step of evaluating visual elasticity of the face of the test subject on the basis of the skin evaluation indexes.
